(19) United States
(12) Patent Application Publication
(10) Pub. No.: US 2008/0221214 A1
(43) Pub. Date: Sep. 11, 2008

(54) Process for the preparation of "Urchym" a urease and alpha-chymotrypsin enzyme inhibitory drug

(76) Inventor: Shahnaz Perveen, Karachi (PK)

Correspondence Address: NATH & ASSOCIATES PLLC, 112 South West Street, Alexandria, VA, 22314, US

(21) Appl. No.: 11/984,141

(22) Filed: Nov. 14, 2007

(30) Foreign Application Priority Data
Nov. 15, 2006 (PK) 1510/2006
Apr. 20, 2007 (GB) 0700816.2

(51) Int. Cl. A61K 31/175; C07C 273/00

(52) U.S. Cl. 514/589; 564/33

(57) ABSTRACT

The present invention relates to the compound N-4-nitrophenyl-N′-4′-nitrophenylurea as a urease and α-chymotrypsin (anti HCV) enzyme inhibitory drug named “Urchym”. N-4-nitrophenyl-N′-4′-nitrophenylurea is prepared and screened for their urease and α-chymotrypsin inhibition effects, the said compound, showed strong urease inhibition (IC₅₀=1.25 μM). We found, that the same compound is also an efficient α-chymotrypsin inhibitor having an IC₅₀ value of 3.15 μM.

### INTRODUCTION

Enzyme inhibition is an important area of pharmaceutical research, since studies in this field have already led to the discovery of wide variety of drugs useful in a number of diseases. Specific inhibitors interact with enzymes and block their activity towards their corresponding natural substrates. Urease inhibitors have recently attracted much attention as potential new anti-ulcer drugs and comprehensive developments in the field were recently reviewed in reference.

Ureases (E.C.3.5.1.5) are enzymes decomposing urea to ammonia and carbamates, and the latter spontaneously decompose to ammonia and carbonic acid. Due to the formation of two molecules of ammonia and carbonic acid, the net effect is an increase in pH. Bacterial ureases are directly involved in the formation of infection stones and contribute to the pathogenesis of pyelonephritis, ammonia, encephalopathy, hepatic coma or urinary catheter encrustation and peptic ulceration. Therefore, strategies based on urease inhibition are now considered as the first line of treatment for infections caused by urease-producing bacteria. In agriculture, high urease activities cause significant environmental and economic problems by releasing abnormally large amounts of ammonia into the atmosphere during urea fertilization. This further induces plant damage primarily by depriving them from their essential nutrient and secondly ammonia toxicity increasing the pH of the soil. To reduce the problems encountered using urea fertilizers, several approaches have been suggested, and the most promising one is to apply urease inhibitors.

Trypsin and chymotrypsin are digestive enzymes and members of a family of enzymes known as serine proteases. They are synthesized as inactive zymogen precursors (trypsinogen and chymotrypsinogen) to prevent unwanted destruction of cellular proteins. The inactive zymogens are secreted into the duodenum, and are converted to the mature, active enzymes by proteolysis to split off a pro-peptide, either in a subcellular compartment or in an extracellelar space where they are required for digestion. Any disturbance of the balance between proteolytic enzymes and their inhibitors, or of the activation process, can result in pancreatits, where premature, intercellular activation of zymogens cause the auto-digestion of the pancreas. Pancreatits carries a 40% risk of pancreatic cancer, a very invasive cancer with high mortality rates. Pancreatic inflammation promotes intensive cell proliferation to regenerate the damaged pancreas, during which the amplification of pathological changes in DNA can occur. The serine proteases contain a uniquely reactive residue at their active site and are inhibited by diisopropylfluorophosphates and serine proteinase inhibitors. Serine proteases, such as chymotrypsin and trypsin, are involved in the destruction of certain fibrous proteins. Chronic infection by hepatitis C virus can lead to the progressive liver injury, cirrhosis, and liver cancer. Viral proteases are an absolute requirement in the life cycle of many viruses and HIV-specific protease inhibitors are designed to target these proteases of HIV-infected patients. Therefore, the search for new effective serine protease inhibitors is still an urgent need for drug development.

### Chemistry

The “Urchym” was prepared by the reaction of 4-nitro phenyl isocyanate in the presences of tertiary arnines e.g., triethyl amine, pyridine or 2,6-lutidine in quantitative yield. The invented compound can also be prepared by the reaction of phenyl isocyanate with tertiary arnines e.g., triethyl amine, pyridine or 2,6-lutidine to give N, N′-diphenylurea, it is then treated with nitrating agent (concentrated nitric and sulfuric acid) to yield N-4-nitrophenyl-N′-4′-nitrophenylurea.

### Biology

Urchym was tested against ureas, using thiourea as a standard inhibitor (IC₅₀ value=21 μM). N-4-nitrophenyl-N′-4′-nitrophenylurea was found to be the most potent urease inhibitor having an IC₅₀ value of 1.25 μM, and is thus superior in activity compared to the standard inhibitor thiourea.

Compound Urchym was also tested for their α-chymotrypsin inhibitory activity and found that it was an excellent α-chymotrypsin inhibitory property with an IC₅₀ value of 3.15±0.14 μM which is far above that of the standard inhibitor chymostatin (IC₅₀=7.00

### Method of Preparation

### Method 1

It is a one pot reaction for the synthesis of N-4-nitrophenyl-N′-4′-nitrophenylurea.
1. 4-nitro phenyl isocyanate is taken in a non polar solvent like 1,4-dioxane, ether, hexane etc., and then tertiary amines e.g., (triethyl amine, pyridine or 2,6 lutidine) is added at 20-50° C. with continuous stiffing. The mole of tertiary amines may be varied within a wide range in order to obtain maximum yield.
2. The progress of the reaction was monitored via TLC. After 5-10 minutes the reaction was completed and the mixture was poured into ice-cold water with continuous stirring. Yellow solid was filtered and gave pure desired N-4-nitrophenyl-N′-4′-nitrophenylurea. Crystallization by ethanol gave pure yellow needles with m.p., 310-312° C.

### Method 2

1. Initially N,N′-biphenyl urea was synthesized by taking phenyl isocyanate in non polar solvent such as ether, hexane, 1,4-dioxane and then treated with tertiary amines e.g., triethyl amine, pyridine and 2,6-lutidine at 20 to 50° C. The concentration of the tertiary amines may be varied within a wide range in order to obtain maximum yield.
2. After completion of reaction the reaction mixture was poured into ice cold water with continuous stiffing, solid was filtered and afforded pure N, N′-biphenyl urea.
3. For the preparation of N-4-nitrophenyl-N′-4′-nitrophenylurea, the nitrating mixture i.e., concentrated nitric and sulfuric acid is added in portion with continuous stirring, in the solution of N, N′-biphenyl urea in non polar solvent e.g., ether, hexane, 1,4-dioxane, during addition the reaction mixture was cooled to 0-10° C.
4. Reflux at 50-60° C. for two hr. The progress of reaction was monitored by TLC.
5. After completion of reaction, the reaction mixture was poured into ice cold water with continuous stirring, yellow solids are separated out and recrystallization from ethanol gave yellow crystalline needles of N-4-nitrophenyl-N′-4′-nitrophenylurea, m.p. 310-312° C.

### EXAMPLE 1(A)

0.2 mol of 4-nitro phenyl isocyanate was taken in a non polar solvent e.g., hexane, ether, 1,4-dioxane and was treated with 1.7 moles of tertiary amines i.e., triethyl amine, pyridine, 2,6-dimethyl pyridine and 2,6-lutidine at room temperature. Progress of reaction was monitored by TLC. After completion of reaction the reaction was poured in ice-water with continuous stirring and solid was separated out, and recrystallization from ethanol gave yellow crystalline needles of N-4-nitrophenyl-N′-4′-nitrophenylurea in 95% yield.

### EXAMPLE 1(B)

0.3 mol of 4-nitro phenyl isocyanate was taken in a non polar solvent e.g., hexane, ether, 1,4-dioxane and was treated with 2.0 moles of tertiary amines i.e., triethyl amine, pyridine, 2,6-dimethyl pyridine and 2,6-lutidine at room temperature. Progress of reaction was monitored by TLC. After completion of reaction the reaction was poured in ice-water with continuous stirring and solid was separated out, and crystallized by a mixture of ethanol and water, gave yellow crystals of N-4-nitrophenyl-N′-4′nitrophenylurea in 95% yield.

### EXAMPLE 2(A)

0.3 mol of phenyl isocyanate in a suitable solvent e.g., hexane, ether, 1,4-dioxane was treated with 2.0 moles of tertiary amines i.e., triethyl amine, pyridine and 2,6-lutidine. After completion of reaction the reaction is poured in ice water and solid of N, N′-diphenylurea was separated out (yield 95%). The N, N′-diphenylurea (0.1 mol) was again dissolved in a non polar solvent like 1,4-dioxane, ether, hexane and then 0.2 mol of nitrating mixture (concentrated nitric and sulfuric acid) was added at 0-10° C. in portion with continuous stirring, reflux at 50-60° C. for 2 hr. After completion of reaction, the reaction mixture was poured into ice-water with stirring, yellow coloured solid was separated out and it is then recrystallized by ethanol (yield 65%).

### EXAMPLE 2(B)

0.4 mol. of phenyl isocyanate in a suitable solvent e.g., hexane, ether, 1,4dioxane was treated with 2.8 moles of tertiary amines i.e., triethyl amine, pyridine and 2,6-lutidine. After completion of reaction the reaction was poured in ice cold water and solid of N, N′-diphenylurea was separated out (yield 95%). 0.1 mol of N, N′-diphenylurea was again dissolved in a non polar solvent like 1,4-dioxane, ether, hexane and then 0.2 mol nitrating mixture (concentrated nitric and sulfuric acid) was added at 40° C. in portion with continuous stirring. It is then reflux at 100° C. for 1 hr. After completion of reaction, the reaction was poured into ice-water with stirring, yellow coloured solid was separated out and it is then recrystallized by ether (yield 85%)

### Urease Assay and Inhibition

Reaction mixtures comprising a 25 μL solution of enzyme (Jack bean urease, Sigma-Aldrich, specific activity 15 EU/mg) and 55 μL of buffer (0.01 M K₂HPO₄3H₂O, 1 MM EDTA 0.01 M LiCl; pH 8.2) containing 100 mM urea were incubated with 5 μL of the test compounds (0.01 μM-1 mM, dissolved in DMSO) at 30° C. for 15 min in 96-well plates. Urease activity was determined by measuring ammonia production using the indophenol method, 45 μL, of phenol reagent (1% w/v phenol and 0.005% w/v sodium nitroprusside) and 70 μL of alkali reagent (0.5% w/v NaOH and 0.1% w/v NaOCI) were added to each well (the final reaction volume was 200 μL). The increasing absorbance at 630 nm was measured after 50 min using a microplate reader (Molecular Device). All reactions were performed in triplicate. The results (change in absorbance per min) were processed using SoftMax Pro software (Molecular Device). Percent inhibitions were calculated using the formula 100−(OD_{test well}/OD_{control}×100). Thiourea was used as the standard inhibitor for urease.

### α-Chymotrypsin Assay and Inhibition

For the determination of α-chymotrypsin inhibitory activity the literature protocol was followed. In brief α-chymotrypsin (9 units/mL in 50 mM Tris-HCl buffer, pH 7.6; Sigma-Aldrich) was pre-incubated with the compound (3.02×10⁻⁵ g/100 μL) for 30 min at 37° C. and then 100 μL of substrate solution (N-succinyl-phenylalanine-p-nitroanilide; 1 mg/mL) in 50 mM Tris-HCl buffer, pH 7.6) was added to start the enzyme reaction. The absorbance of released p-nitroaniline was continuously monitored at 410 nm until a significant color change had achieved. All reactions were performed in triplicate. Percent inhibitions were calculated using the formula 100-(OD_{test well}/OD_{control}×100). Chymostatin was used as standard inhibitor for α-chymotrypsin.

1. The compound N-4-nitrophenyl-N′-4-nitrophenylurea “Urchym” useful as enzyme urease and α-chymotrypsin inhibitory drug.

2. The compound as claimed in claim 1 which prepared and screened shows strong urease inhibition having IC₅₀ value of 1.25 μM.

3. The compound as claimed in claim 1 which shows strong α-chymotrypsin inhibition having an IC₅₀ value of 3.15 μM.

4. The compound as claimed in claim 1 useful as enzyme urease and α-chymotrypsin inhibitory drug having m.p. 310-312° C.

5. The compound “Urchym” as claimed in claim 1 tested against for urease using thiourea as standard inhibitor (IC₅₀ value=21) was found most potent urease inhibitor.

6. The compound “Urchym” as claimed in claim 1 also tested for the α-chymotrypsin inhibitory activity was found excellently efficient with an IC₅₀ value of 3.15+0.14 μM.

7. The compound as claimed in claim 2 useful as enzyme urease and α-chymotrypsin inhibitory drug having m.p. 310-312° C.

8. The compound as claimed in claim 3 useful as enzyme urease and α-chymotrypsin inhibitory drug having m.p. 310-312° C.

9. The compound “Urchym” as claimed in claim 2 tested against for urease using thiourea as standard inhibitor (IC₅₀ value=21) was found most potent urease inhibitor.

10. The compound “Urchym” as claimed in claim 3 tested against for urease using thiourea as standard inhibitor (IC₅₀ value=21) was found most potent urease inhibitor.

11. The compound “Urchym” as claimed in claim 4 tested against for urease using thiourea as standard inhibitor (IC₅₀ value=21) was found most potent urease inhibitor.

12. The compound “Urchym” as claimed in claim 2 also tested for the α-chymotrypsin inhibitory activity was found excellently efficient with an IC₅₀ value of 3.15+0.14 μM.

13. The compound “Urchym” as claimed in claim 3 also tested for the α-chymotrypsin inhibitory activity was found excellently efficient with an IC₅₀ value of 3.15+0.14 μM.

14. The compound “Urchym” as claimed in claim 4 also tested for the α-chymotrypsin inhibitory activity was found excellently efficient with an IC₅₀ value of 3.15+0.14 μM.
